(19) United States
(12) Patent Application Publication
(10) Pub. No.: US 2009/0326489 A1
(43) Pub. Date: Dec. 31, 2009

(54) Applicator for a water jet separating apparatus

(76) Inventors: Arnd Kensy, Michendorf (DE); Konrad-Wenzel Winkler, Warin (DE); Frank Niklas, Lubz (DE); Thomas Peulecke, Schwerin (DE); Andreas Runow, Schwerin (DE); Fred Behnert, Dummer (DE); Gernot Schlee, Schwerin (DE)

Correspondence Address: WALTER OTTESEN, PO BOX 4026, GAITHERSBURG, MD, 20885-4026, US

(21) Appl. No.: 12/457,581

(22) Filed: Jun. 16, 2009

Related U.S. Application Data
(63) Continuation of application No. PCT/DE2007/002057, filed on Nov. 14, 2007.

(30) Foreign Application Priority Data
Dec. 16, 2006 (DE) 20 2006 018 986.6

(51) Int. Cl. A61M 1/00

(52) U.S. Cl. 604/319

(57) ABSTRACT

In order to provide a work area that is as large as possible, and to shield off the environment, and in order to simultaneously provide for a mobile application, the discharge nozzle (11) and the suction opening (12) are disposed in a protective cover (6, 6′, 6″, 6′″). The protective cover (6, 6′, 6″, 6′″) and the body parts of the patient conjointly form a defined vacuum-sealed work chamber (8). The protective cover is made of a transparent material.

### CROSS REFERENCE TO RELATED APPLICATIONS

This application is a continuation application of international patent application PCT/DE 2007/002057, filed Nov. 14, 2007, designating the United States and claiming priority from German application 20 2006 018 986.6, filed Dec. 16, 2006, and the entire content of both applications is incorporated herein by reference.

### FIELD OF THE INVENTION

The invention relates to an applicator for a water jet separating apparatus especially for treating wounds and abscesses.

### BACKGROUND OF THE INVENTION

Wounds and abscesses are injuries to the skin and heal best when: an adequate blood flow is present for self cleaning; the bacteria, which have developed in the ulcer, are destroyed or are removed; and, the upper epidermis of the tissue is configured to have sharp edges to facilitate better healing. For smaller wounds, it is only necessary to clean and disinfect in order to satisfy these conditions.

Larger wounds and ulcers have, as a rule, irregular wound edges and ulcers have a greater proportion of necrotic tissue. The necrotic tissue mostly extends over a large area and often extends deeply which hinders the healing process and greatly increases the danger of an infection. An especially dangerous and treatment intensive ulcer is the leg ulcer or lower leg ulcer (ulcus cruris).

Ulcers are, as a rule, mechanically treated in that the necrotic tissue is scraped out or cut away with the aid of a curette or a scalpel. Penetration extends into healthy tissue to improve bleeding.

This method is, however, not very pleasant for the physician conducting the procedure and is not adequately free of pain for the patient.

For this application, there exists the need for a water jet separating technology which has been already widely introduced in medical technology.

This water jet separating technology basically includes: a pressure generating unit having a discharge nozzle for a separating liquid jet; a vacuum generating unit having one or several suction openings for a suction flow; and, an applicator which is adapted to the special case at hand. In this applicator, the discharge nozzle for the separating liquid jet and the suction openings for the suction flow are configured and arranged in a special manner. This water jet separating technology is used successfully and invasively always where body cavities are present wherein the separating liquid can locally collect so that it can be drawn away by suction with the separated tissue parts. This condition is not present for the treatment of wounds in general and especially not for the treatment of ulcers. The use of conventional water jet separating technology is therefore not suitable in these cases because the separating liquid and the pathogenic microorganisms from the ulcer could flow uncontrolled into the surroundings of the area of surgery. This endangers the patient and the personnel.

U.S. Pat. No. 5,941,859 discloses a wound irrigation apparatus having a liquid feedback and having a bell-shaped protective shield. Within this protective shield, there is a discharge nozzle for a liquid medication and a suction stub for the exited liquid medicine. The protective shield covers the wound during the treatment and thereby protects, the surgeon from liquid enriched with bacteria. This protection is, however, only slight because no adequate covering of the work region is present. Furthermore, the wound irrigation apparatus is only provided for irrigating and cleaning a wound and a surgical intervention for treating the wound is not possible.

In the publication of Guthke et al entitled “Wundreinigung bei Ulcus cruris-eine Indikation für den Hochdruckwasserdissektor”, Zentralblatt Haut 1994:164; pages 181 and 182, a treatment tent, which shields against the surroundings having a discharge lock of the Medaxis Company is described which encloses the entire work area for the surgery and prevents an uncontrolled propagation of microorganisms. This discharge lock does not correspond to present day requirements because it greatly limits the manipulability of the surgeon and because it requires an unusually high suction power in order to withdraw by suction the content of the large-volume discharge lock, this content comprising air, water and tissue parts. The drive units must be designed correspondingly large and powerful and therefore it is not possible to use this water jet separating apparatus for the outpatient treatment of wounds.

There are a series of applicators known wherein the discharge nozzle of the pressure line and the inlet opening of the suction line are so arranged toward each other that the exiting water jet together with the separated tissue parts enter directly into the suction opening.

U.S. Pat. No. 6,290,670 discloses an applicator wherein the discharge nozzle is configured as a nozzle ring having a peripherally-extending nozzle slit directed radially. This slit is so directed and the pressure of the liquid jet and the suction flow are so matched to each other that a liquid membrane is formed which dips into the suction tube with this liquid membrane separating the tissue.

U.S. Pat. Nos. 6,375,635, 6,960,182 and 7,122,017 describe an applicator wherein the discharge nozzle of the water jet and the inlet opening of the suction line are positioned at a specific distance relative to each other. The distance is so selected that an additional suction force is generated in accordance with the venturi principle and this suction force improves the removal of the separated tissue. In special cases, a separate suction unit is not needed.

In contrast, German published patent application 40 18 736 discloses an applicator which, at the distal end of the suction tube, has a nozzle ring having two discharge nozzles for the separating fluid which are directed radially and into the interior of the suction tube.

All these applicators do avoid an uncontrolled scattering of the exiting separating liquid because of the direct proximity of the discharge nozzle and the suction opening; however, they have in common the disadvantage that they define a very narrow work area. The distance between the discharge nozzle and the suction opening is, however, narrow and related to function and an increase of this distance is only possible to a limited extent because the size of the suction opening and the intensity of the suction flow can then no longer ensure a trouble-free take-up of the entire liquid flow. These applicators are exclusively provided for the invasive use and therefore are unsuitable for a debridement of open and large area wounds and ulcers.

### SUMMARY OF THE INVENTION

It is an object of the invention to provide an applicator which ensures the largest possible work area and shields the surroundings while at the same time being suitable for mobile use.

The applicator of the invention is for a water jet separating apparatus including an apparatus for treating a wound or an ulcer. The applicator includes: a handpiece; a protective cover connected to the handpiece and being provided for placement on a body part of a patient over the wound or ulcer; a pressure line having a discharge nozzle arranged in the protective cover; a suction line having an opening likewise arranged in the protective cover; the protective cover and the body part conjointly defining a vacuum-closed work chamber; and, a vacuum sensor for monitoring the vacuum in the work chamber.

The new applicator eliminates the above-mentioned features of the state of the art. With the invention, all the known advantages of the water jet separation are effected with these advantages being especially that a cut can be made deep into the healthy tissue in a protected manner which can be tolerated by the patient. These advantages include, for example, the following: the use of a dynamic and pulsed water jet; the use of an abrasive separating means; the use of an antibiotic, antiseptic medium; or, the use of a temperature-controlled separating means. Here, it is especially advantageous when a vacuum is generated in the protective cover via the suction line which vacuum is preferably pulsating and stimulates the tissue. This facilitates the healing process. The special advantage of the new applicator lies in that the intervention takes place in a work chamber closed off with a vacuum therein. This draws the tissue parts by suction into the work chamber and prepares the tissue parts in an advantageous manner for the separating operation. The vacuum, however, ensures also that the blood flow of the wound or the ulcer is increased which advantageously affects the cleaning of the wound or ulcer. A special advantage, however, is that the propagation of pathogenic microorganisms is reliably prevented for the protection of the surgeon and the surroundings. This protection takes place because of the vacuum-conditioned sealing of the work chamber during the surgical procedure. The protection results, however, in that the separating liquid is controlled by the vacuum and can only discharge when the sealing of the work chamber is ensured. An additional shield is provided during the switch-off phase by a special sealing element having a novel catch flap.

The new applicator also provides a work area of increased size because the protective cover can be dimensioned, configured and arranged independently of the arrangement of the discharge nozzles and the suction openings. In this way, larger wounds or ulcer surfaces can be treated. Should a repositioning nonetheless be required, this is easily realized by the surgeon with the control of the vacuum via the bypass opening in the suction flow.

### BRIEF DESCRIPTION OF THE DRAWINGS

The invention will now be described with reference to the drawings wherein:

FIG. 1 is section view of an applicator according to a first embodiment of the invention;

FIG. 2 is a side elevation view of the applicator of FIG. 1;

FIG. 3 is a plan view of the applicator of FIG. 1;

FIG. 4 is a section view through an applicator according to a second embodiment of the invention;

FIG. 5 is a side elevation view of the applicator of FIG. 4;

FIG. 6 is a plan view of the applicator of FIG. 4;

FIG. 7 is a plan view of the applicator of FIG. 4 having a modified nozzle arrangement;

FIG. 8 is a longitudinal section view taken through an applicator according to a third embodiment of the invention;

FIG. 9 is a section view taken through the applicator of FIG. 8;

FIG. 10 is a longitudinal section view taken through a modified applicator according to the third embodiment of the invention of FIG. 8;

FIG. 11 is another longitudinal section view of the modified applicator of FIG. 10;

FIG. 12 is a side elevation view of an applicator according to a fourth embodiment of the invention; and,

FIG. 13 is a side elevation view of an applicator modified compared to the applicator of FIG. 12.

### DESCRIPTION OF THE PREFERRED EMBODIMENTS OF THE INVENTION

The applicator 1 belongs to a surgical water jet separating apparatus which comprises a liquid separating unit for separating a biological structure and a corresponding suction unit. This water jet separation apparatus is generally well known in medical technology and therefore needs no further description. The water jet separating apparatus accordingly comprises a supply vessel for a sterile liquid, a pressure pump and an injection line and the suction unit comprises a collecting vessel for the liquid and the separated tissue parts as well as a suction pump and an suction line. The injection line of the water jet separating apparatus and the suction line of the suction unit open in common in the applicator 1.

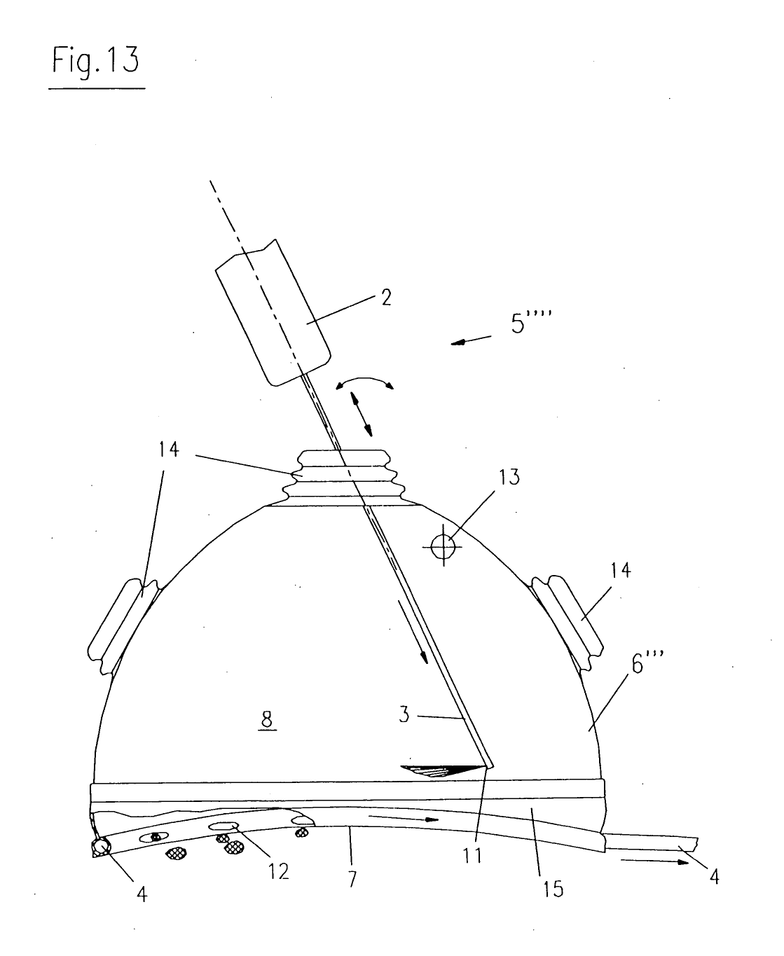

The applicator 1 of all embodiments shown except for FIG. 13 comprises a handpiece 2 which holds together the pressure line 3 for the exiting water separating jet and the suction line 4 for the liquid taken up and the separated tissue parts and leads the pressure line and suction line to a work probe 5 disposed at the distal end of the applicator 1. The pressure line 3 and the suction line 4 are arranged separately from each other and directly adjacent and axially parallel to each other. A bypass opening 13 is disposed in the suction line 4 in the handle region of the handpiece 2.

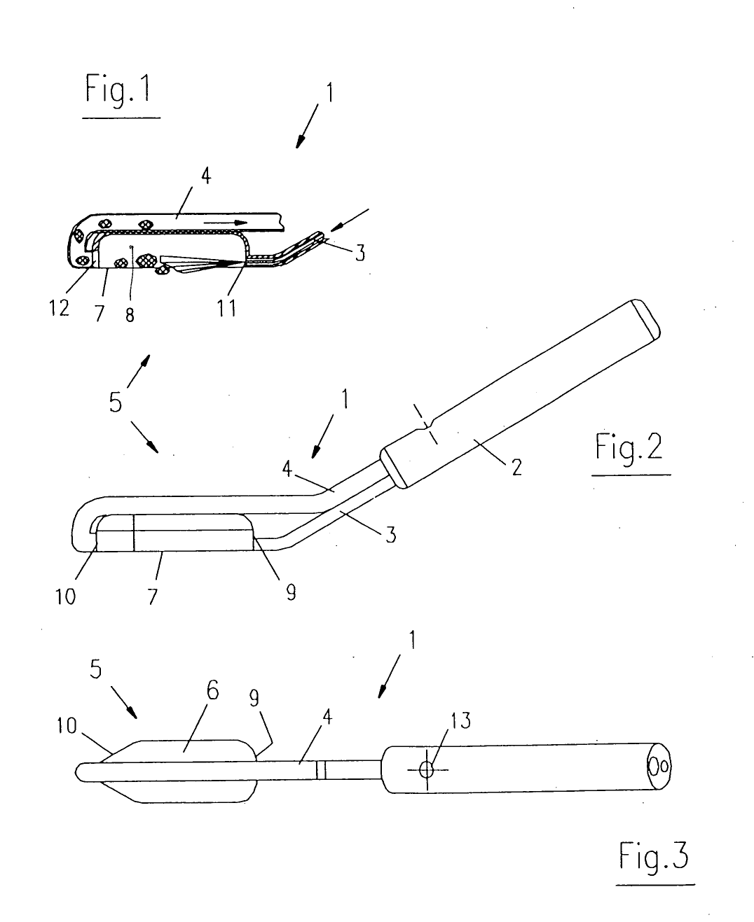

In a first embodiment as shown in FIGS. 1 to 3, the work probe 5 has a flat protective cover 6 open downwardly and closed upwardly and to the sides. The protective cover 6 has a peripherally-extending sealing edge 7 on its open side facing downwardly. The sealing edge 7 can be adapted to the greatest extent possible to the shape of the body of the patient and is preferably equipped with a special sealing material which permits an airtight closure to the body surface. In this way, a hermetically closed off work chamber 8 is formed. The protective cover 6 comprises a transparent material in order to permit viewing into the work chamber 8 from the outside and therefore onto the work area. This protective cover 6 is preferably elongated and has a lateral surface 9 close to the handpiece 2 and a lateral surface 10 remote with respect to the handpiece 2. The pressure line 3 for the water jet opens laterally into the lateral surface 9 close to the handpiece 2 and forms a discharge nozzle 11 there. This discharge nozzle 11 is preferably a flat nozzle. The suction line 4 is guided above the protective cover 6 to the lateral surface 10 remote with respect to the handpiece 2. The suction line 4 opens into the lateral surface 10 at the lowest location of the protective cover 6 and has a suction opening 12 in this region. The suction opening 12 lies opposite the discharge nozzle 11 in the same plane and therefore in the effective region of the discharge nozzle 11. The protective cover 6 is configured in the form of a funnel in the region of the suction opening 12 to improve the take up of the water separating jet and the separated tissue parts. This protective cover 6 hermetically shields the working area of the surgeon relative to the surroundings.

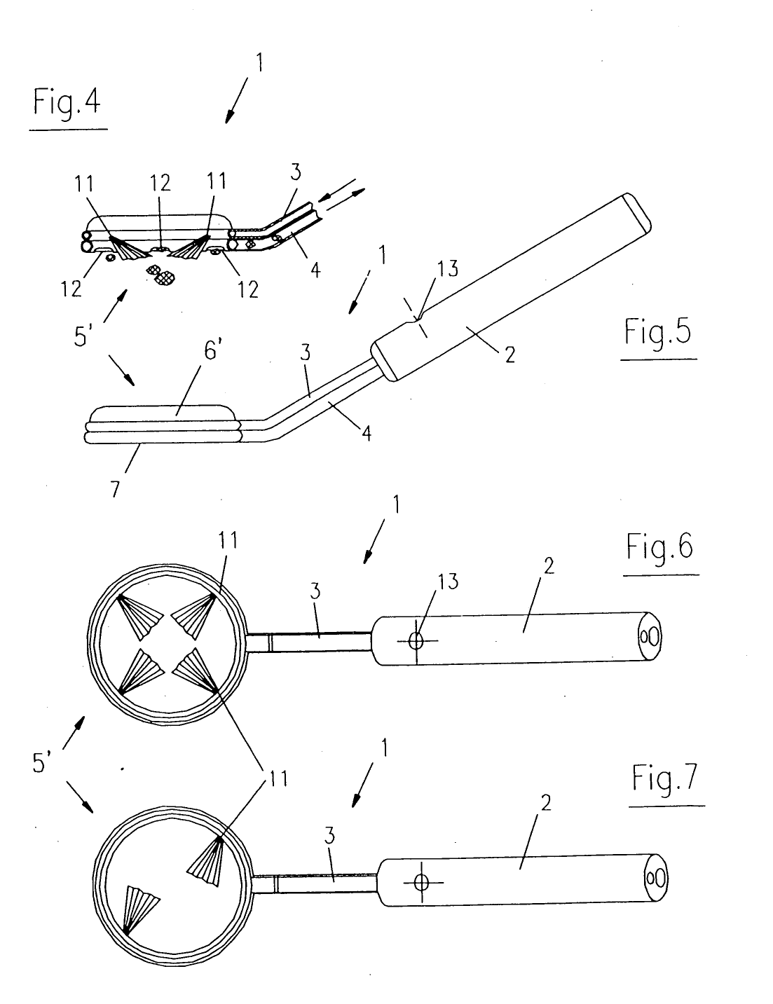

As shown in FIGS. 4 to 7, in the second embodiment, the work probe 5′ has, in the same manner, a handpiece 2 having a pressure line 3, a suction line 4 and a protective cover 6′ having a sealing edge 7. The pressure line 3 and the suction line 4 are, in turn, led laterally to the protective cover 6′. The protective cover 6′ is configured to be round when viewed in cross section and the pressure line 3 and the suction line 4 are guided on the periphery of the protective cover 6′. The peripherally-extending segment of the suction line 4 is disposed at the lowest plane and the pressure line 3 is disposed in the plane of the protective cover 6′ lying thereabove. For this purpose, preferably several suction openings 12 are uniformly distributed in the peripherally-extending segment of the suction line 4. As shown in FIG. 6, the pressure line 3 lying thereabove has four discharge nozzles 11, which are arranged uniformly offset, so that always two of the discharge nozzles 11 lie opposite each other. The discharge nozzles 11 are directed toward the lower-lying plane wherein the suction openings 12 are disposed. The four or even more discharge nozzles 11 thereby form a focus at the center of the protective cover 6′. The discharge openings 12 are preferably provided as an even number.

As shown in FIG. 7, only two of the discharge nozzles 11 are provided which lie opposite each other in a plane but are directed laterally past each other. This relationship is so selected that a tissue part, which is struck by the separating water jets of the two discharge nozzles 11, is set into rotation or spin. This supports the separation.

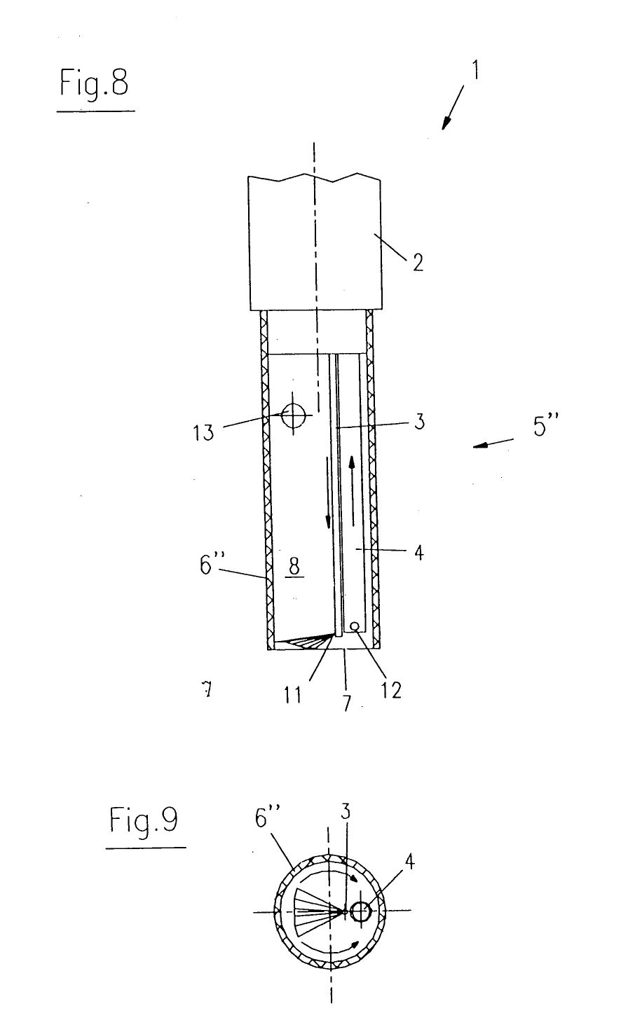

In the embodiment of FIGS. 8 and 9, the work probe 5″ has a cylindrical protective cover 6″ which is tightly connected to the handle 2. The protective cover 6″ accommodates and surrounds the pressure line 3 and the suction line 4 in the distal region of the applicator 1 in their longitudinal dimension. The protective cover 6″ is configured to be open at the distal end so that a circular sealing edge 7 is formed. The pressure line 3 and the suction line 4 are again arranged axially parallel to each other and to the cylindrical protective cover 6″. Together, the pressure line 3 and the suction line 4 are, in a special manner, arranged off center to the cylindrical protective cover 6″ in such a manner that the suction line 4 is axis remote and in direct proximity to the interior wall surface of the cylindrical protective cover 6″ and the pressure line 3 is disposed on the axis-near side of the suction line 4.

At the distal end, the suction line 4 has an end face suction opening 12 and, selectively, additional suction openings 12 which can be arranged distributed over the periphery. Correspondingly, the suction line 4 ends in its length at a predetermined distance above the sealing edge 7 of the protective cover 6″ in order to maintain the end face suction opening 12 open. The pressure line 3 has a discharge nozzle 11 which is arranged at the outermost distal end and is arranged radially and is directed in a direction away from the suction line 4.

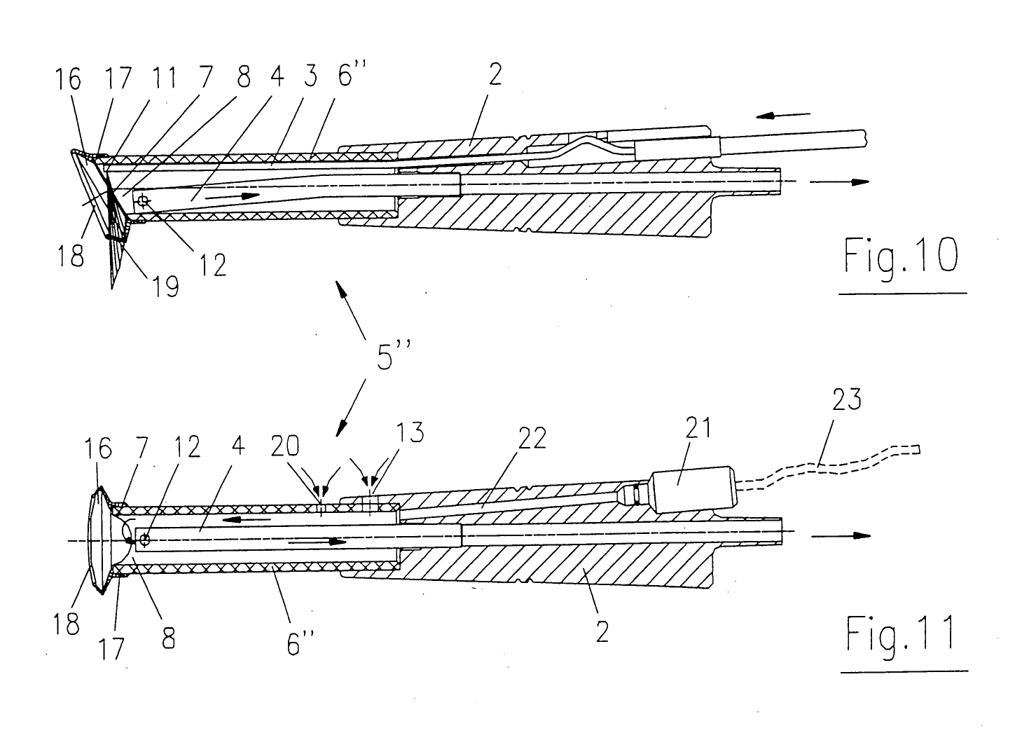

The modified work probe 5″ of the third embodiment is shown in FIGS. 10 and 11 and has, in turn, a cylindrical protective cover 6″ which is made of a transparent material and which accommodates and encloses the pressure line 3 and the suction line 4 along the length in the distal region of the applicator 1. The protective cover 6″ is connected tightly to the handle 2. The pressure line 3 and the suction line 4 are arranged at a radial distance with respect to each other and to the cylindrical protective cover 6″ in such a manner that the ends of the pressure line 3 and the suction line 4 are arranged with respect to each other at the distal region at a largest radial distance. The pressure line 3 is closed at the end face with the discharge nozzle 11 being arranged radially at the outermost end of the pressure line 3. The discharge nozzle 11 is preferably a flat nozzle. The discharge nozzle 11 is directed in the direction of the end of the suction line 4. This end of the suction line 4 has an end face suction opening 12 and, selectively, further radial suction openings 12. The protective cover 6″ is, in turn, configured open at the distal end.

In the present embodiment, the end of the protective cover 6″ is beveled so that an oval sealing edge 7 is formed. A special sealing element 16 is seated on this sealing edge 7. The sealing element 16 is made of a flexible material and therefore is adaptable to different body shapes of the patient.

This sealing element 16 has a proximal holding ring 17 which ensures the connection to the cylindrical protective cover 6″ and a distal sealing lip 18 which lies on the body surface of the patient. In a special manner, the distal sealing lip 18 is equipped with a catch flap 19 movable in the axial direction in the region of the suction openings 12 of the suction line 4. The catch flap 19 is movable between an unfolded position in the rest position as shown in FIG. 10 and an in-folded position (not shown) in the active use position.

In the region of the handpiece 2, the protective cover 6″ likewise has a first bypass opening 13 which is held open or closed by the hand of the surgeon and is so dimensioned that, in the open state, the suction flow is conducted via the bypass opening 13 and therefore a build up of suction forces at the distal end is prevented. A second bypass opening 20 is disposed in the vicinity of the first bypass opening 13. The second bypass opening is so dimensioned to match with the power of the vacuum pump that a defined suction flow having a specific suction force adjusts for a complete sealing at the sealing element 16 and a closed bypass opening 13.

In the handpiece 2 of the applicator 1, a vacuum sensor 21 is also provided which, on the one hand, is connected via a pressure sensor line 22 to the work chamber 8 and, on the other hand, via a signal line 23 to the liquid conveying pump. The vacuum sensor 21 is so configured and so coupled to the liquid conveying pump that the liquid conveying pump is only capable of pumping at a predetermined vacuum pressure range. A pumping of liquid and therefore a discharge of liquid from the discharge nozzle 11 are not possible outside of this vacuum pressure difference.

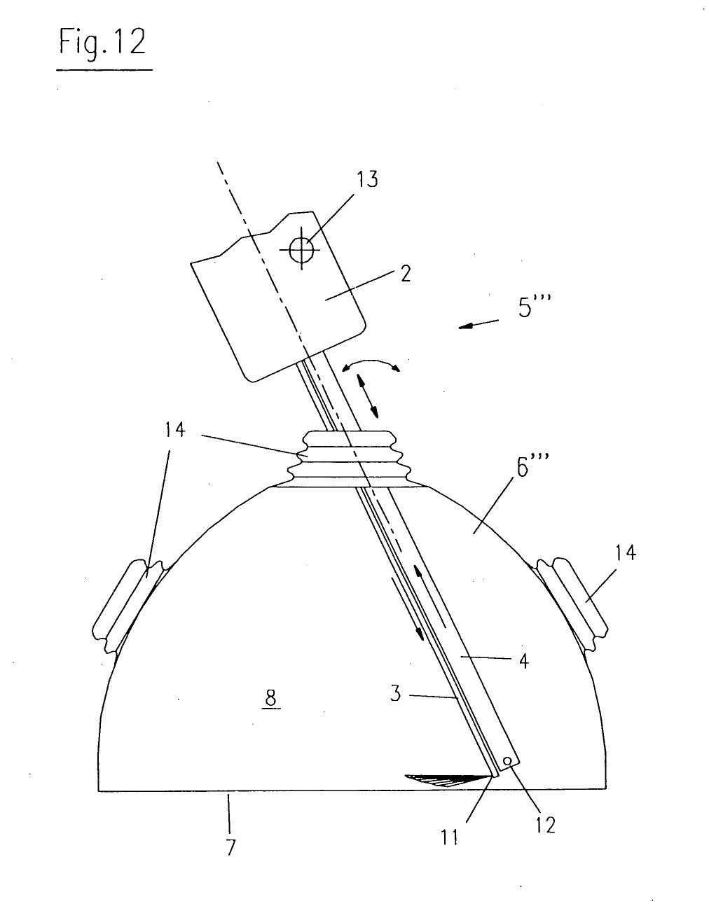

According to FIG. 12, the work probe 5″ is equipped with a protective cover 6′″, which is configured as a bell and which is again open at the lower region and is equipped with a circular sealing edge 7. An opening is disposed in the bell dome of the protective cover 6′″. The pressure line 3 and the suction line 4 are passed through this opening while lying axially parallel one next to the other. In this opening in the bell dome, a flexible holding and sealing element 14 is seated which holds the pressure line 3 and the suction line 4 and seals off, with respect to the ambient, the intermediate space between the protective cover 6′″ and the pressure line 3 and the suction line 4. The holding and sealing element 14 is connected tightly to the protective cover 6′″ and is slideably configured relative to the pressure line 3 and the suction line 4. In this way, the pressure line 3 and the suction line 4 are adjustable in elevation as well as in angular position so that the distal ends of the pressure line 3 and the suction line 4 can together reach each point of the work surface within the protective cover 6′″. In addition to the flexible holding and sealing element 14 in the bell dome or alternatively thereto, additional openings, which are each closed with a holding and sealing element 14, can be provided for selectively passing through the pressure line 3 and the suction line 4. In this way, the accessibility to the work area of the surgeon can be improved.

The distal ends of the pressure line 3 and the suction line 4 are, in turn, equipped with a radial discharge nozzle 11, which is directed away from the suction line 4, and an end face suction opening 12 and, selectively, further radial suction openings 12.

According to FIG. 13, the protective cover 6′″ has a peripherally-extending sealing element 15 in the region of its sealing edge 7. The sealing element 15 is secured so that it can be exchangeable on the protective cover 6′″ and the sealing edge 7 is adapted to specific body parts of the patient. This sealing element 15 can be exchanged for another sealing element 15 having another contour of the sealing edge 7. The sealing element 15 can also comprise a flexible material and therefore be configured to be self adapting. In the sealing element 15, the suction line 4, which is likewise configured to be peripheral, is integrated and is directly guided away. The suction line 4 has, in its peripherally-extending region, several suction openings 12. Correspondingly, only the pressure line 3 is guided through the holding and sealing element 14 whereby also only this pressure line 3 is manipulated during surgical procedures.

During operation of the water jet separating apparatus, a predetermined water pressure adjusts in the pressure line 3 which forms a corresponding preferably flat water separating jet in combination with the exit nozzle 11. The suction line 4 is first without pressure because the bypass opening 13 is opened and so generates an ancillary suction flow. With the closure of this bypass opening 13 by the surgeon, an underpressure builds up in the work chamber 8 of the protective cover (6, 6′, 6″, 6′″) which lifts the tissue disposed in the region of the work probe 5, 5′, 5″, 5′″, 5″″ and presses it against the sealing edge 7 of the protective cover 6, 6′, 6″, 6′″. In this way, the work area is hermetically sealed with respect to the surroundings so that no water and therefore no bacteria can exit from the work area. At the same time, the tissue to be separated by the force of the suction flow within the protective cover 6 is lifted and stretched and is brought into a favorable position for the separating operation. The water jet does its work in that it penetrates in an intelligent manner between the tissue cells and expands these cells and further tensions and separates the cells one from the other. Tissue cells are hardly damaged thereby. The suction flow entrains the separated tissue cells together with the collected water and moves the same through the suction openings 12 to the collecting vessel.

In the first embodiment of FIGS. 1 to 3, the separating jet and the suction flow within the protective cover 6 are directed in the same direction so that the two forces superpose and a high separation and suction effect is obtained.

The applicator 1 of the second embodiment of FIGS. 4 to 7 develops several separating water jets which lie opposite each other. In this way, the separating water jets are focused whereby the tissue, which is to be separated, is undermined from all sides and disintegrated in the focus. In the arrangement of the discharge nozzle 11 according to FIG. 7, an additional rotational pulse is imparted to the tissue which is to be separated and this further facilitates the separation.

In the third and fourth embodiments of FIGS. 8, 9, 12 and 13, the water separating jet is directed away from the suction opening 12. The separating water jet is then picked up by the round inner wall of the protective cover (6″, 6′″) and deflected so that the water separating jet glides along the inner wall and is guided to the suction opening 12.

After the successful removal of devitalized tissue, the pressure generation for the water jet is switched off and the bypass opening 12 is opened by the surgeon so that the suction flow relaxes and the tissue is again exposed which was drawn by suction onto the sealing edge 7 of the protective cover (6, 6′, 6″, 6′″). Thereafter, the applicator 1 is moved into the next work area and the applicator 1 is taken into service anew.

This repositioning of the applicator 1 within the wound or the ulcer is rendered essentially unnecessary in the embodiment of FIGS. 12 and 13 because the bell-shaped protective cover 6′″ is configured to have a large volume so that it covers the wound or the ulcer, as a rule, in totality. The processing here takes place step by step via the vertical lifting and the horizontal deflection of the bundled pressure line 3 and suction line 4 within the work chamber 8.

The modified applicator 1 in the third embodiment of FIGS. 10 and 11 has further functions and advantages. The beveled course of the distal sealing edge 7 provides an oval work surface on the body of the patient and therefore a largest possible distance between the discharge nozzle 11 and the suction openings 12. A work position having greater manipulability is imparted to the applicator 1 with this beveled sealing edge 7. With the closing of the first bypass opening 13, a suction flow flows via the second bypass opening 20, the work chamber 8 and the suction openings 12 in the suction line 4. This suction flow at first determines the magnitude of the vacuum and, during the water jet separation, provides for a supporting entraining effect for the separated tissue. The body tissue of the patient is pulled against the sealing edge 7 in a sealing manner because of the suction power of the vacuum pump on the one hand and because of the throughflow resistance at the second bypass opening 20 on the other hand. At special body locations of the patient, which do not permit a tissue displacement of this kind or do not permit it adequately, as is the case, for example, in the region of limbs, the vacuum sucks the flexible sealing element 16 into body depressions and ensures thereby also in these cases a functionally-caused sealing. This vacuum pressure is monitored by the vacuum sensor 21. After a predetermined vacuum pressure has adjusted in the work chamber 8, the vacuum sensor 21 signals a control command for activating the liquid pump. The generated water jet then performs its work in the intelligent manner already described.

After the successful removal of devitalized tissue, the surgeon opens the bypass opening 13 so that the vacuum relaxes and the sealing element 16 releases from the surface of the body. The vacuum sensor 21 detects this pressure change in the work chamber and issues a control signal to the liquid pump in order to immediately permit the same to be switched off. This control signal simultaneously initiates a short reversal of the pumping direction in order to minimize an afterrunning of the liquid pump and therefore a limited further pumping action. The surgeon therefore lifts the applicator 1 from the body surface of the patient directly after the opening of the first bypass opening 13 in order to be able to seat the same at the next body location. Because of the sensor-supported on and off switching of the liquid pump, an uncontrolled exiting of liquid laced with bacteria into the ambient is prevented. An additional protection against the unwanted exiting of contaminated liquid occurs via the special function of the sealing element 16. Because of the natural movement of the surgeon, the upper portion of the sealing edge 7 with the discharge nozzle 11 first releases when the applicator 1 is lifted while the lower portion of the region of the suction openings 12 still remains on the surface of the body. In this way, the catch flap 19 opens with this flap taking up a pressure liquid jet which is present for a short time against expectation and is deflected into the interior of the work chamber 8. An exiting of the pressure liquid jet from the region of the work chamber 8 in the proximity of the surgeon is thereby prevented. After the repositioning of the applicator 1, this function repeats at another body location.

It is understood that the foregoing description is that of the preferred embodiments of the invention and that various changes and modifications may be made thereto without departing from the spirit and scope of the invention as defined in the appended claims.

### LIST OF REFERENCE NUMERALS

1 Applicator
2 Handpiece
3 Pressure line
4 Suction line
5 Work probe
6 Protective cover
7 Sealing edge
8 Work chamber
9 Near lateral surface
10 Remote lateral surface
11 Discharge nozzle
12 Suction opening
13 Bypass opening
14 Flexible holding and sealing element
15 Sealing element
16 Sealing element
17 Holder
18 Sealing lip
19 Catch flap
20 Second bypass opening
21 Vacuum sensor
22 Pressure sensor line
23 Signal line

What is claimed is:

1. An applicator for a water jet separating apparatus including an apparatus for treating a wound or an ulcer, the applicator comprising:
a handpiece;
a protective cover connected to said handpiece and being provided for placement on a body part of a patient over the wound or ulcer;
a pressure line having a discharge nozzle arranged in said protective cover;
a suction line having an opening likewise arranged in said protective cover;
said protective cover and said body part conjointly defining a vacuum-closed work chamber; and,
a vacuum sensor for monitoring the vacuum in said work chamber.

2. The applicator of claim 1, wherein said protective cover is made of a transparent material.

3. The applicator of claim 1, wherein said discharge nozzle is directed toward said suction opening.

4. The applicator of claim 3, wherein said protective cover has a near lateral wall near said handpiece and a remote lateral wall disposed opposite said near lateral wall; said discharge nozzle is disposed radially on said near lateral wall and said suction opening is disposed radially on said remote lateral wall.

5. The applicator of claim 1, wherein said protective cover has a peripheral wall defining a periphery; said discharge nozzle is disposed on said periphery in an upper plane; and, said suction opening is disposed on said periphery in a lower plane below said upper plane.

6. The applicator of claim 5, wherein said discharge nozzle is a first discharge nozzle for discharging a first water jet; and, wherein said applicator further comprises a second discharge nozzle for discharging a second water jet; and, said first and second water jets conjointly define a focus.

7. The applicator of claim 5, wherein said discharge nozzle is a first discharge nozzle for discharging a first water jet; and, wherein said applicator further comprises a second discharge nozzle for discharging a second water jet; and, said first and second discharge nozzles are disposed so as to cause said first and second water jets to meet at a spacing from each other thereby generating a center spin in said protective cover.

8. The applicator of claim 3, wherein said protective cover is configured to have a tubular configuration enclosing said pressure line and said suction line along a length thereof; said protective cover has a distal region; and, said pressure line and said suction line are arranged in said protective cover so as to be off center with respect thereto in such a manner that said discharge nozzle and said suction opening are at the greatest radial distance from each other in said distal region.

9. The applicator of claim 8, wherein said protective cover is configured as a circular cylinder.

10. The applicator of claim 1, wherein said discharge nozzle is disposed radially and away from said suction opening and is directed against said protective cover.

11. The applicator of claim 10, wherein said protective cover is configured to have a tubular configuration defining an axis and enclosing said pressure line and said suction line along at least a portion of the lengths thereof; said protection cover has a distal end whereat said protective cover is configured to be open; said pressure line and said suction line are disposed in said protective cover so as to be off center with respect thereto in such a manner that said suction line is remote from said axis and has an axis-near side and said pressure line is disposed on said axis-near side of said suction line.

12. The applicator of claim 11, wherein said protective cover is configured to be cylindrical.

13. The applicator of claim 10, wherein said protective cover is configured to have a bell shape and encloses said pressure line and said suction line along at least a portion of the respective lengths thereof; and, wherein said applicator further comprises means for articulately and displaceably journalling said pressure line and said suction line within said protective cover.

14. The applicator of claim 13, wherein said pressure line and said suction line are articulately and displaceably journalled in parallel within said protective cover.

15. The applicator of claim 13, wherein said journalling means is a joint arranged in said protective cover; and, said joint is configured as a flexible holding and sealing element which can be closed when not in use.

16. The applicator of claim 1, wherein said protective cover has a sealing element defining a sealing edge and being adaptable to a body part of a patient.

17. The applicator of claim 16, wherein said sealing element has a catch flap in the jet region of said discharge nozzle which folds into said work chamber while said applicator is in use and folds out in the direction of the surface of said body part when said applicator is lifted from the location of treatment.

18. The applicator of claim 1, wherein a pulsating vacuum is generated via said suction line in said work chamber which stimulates the tissue.

19. The applicator of claim 1, wherein said water jet separating apparatus includes a pump for moving liquid; and, wherein said applicator further comprises a vacuum sensor connected to said protective cover and a signal lead; and, said vacuum sensor is so connected to said pump via said signal lead that said pump is capable of moving liquid only within a predetermined vacuum pressure range which is evaluated via said vacuum sensor.

20. The applicator of claim 19, wherein said vacuum sensor is connected to said pump in such a manner that the pumping direction of said pump reverses for a short time when said pump is switched off.
